(19) United States
(12) Patent Application Publication
(10) Pub. No.: US 2019/0320698 A1
(43) Pub. Date: Oct. 24, 2019

(54) HYDROLYZED PECTIN AND USEOF THE SAME

(71) Applicant: International Flavors & Fragrances Inc., New York, NY (US)

(72) Inventors: Daniel Kaiping Lee, Morganville, NJ (US); Ying Yang, Holmdel, NJ (US); Ronald Gabbard, Farmingdale, NJ (US)

(21) Appl. No.: 15/958,000

(22) Filed: Apr. 20, 2018

(51) Int. Cl. A23L 27/24; C12P 19/14; A23L 29/231; A23L 27/00; A23L 33/24

(52) U.S. Cl. CPC A23L 27/24; C12P 19/14; A23L 29/231; A23V 2002/00; A23L 27/72; A23L 33/24; A23L 27/80

(57) ABSTRACT

A process of hydrolyzing a pectin. The process involves hydrolyzing a pectin in a reaction mixture containing a pectinase to obtain a hydrolyzed pectin. Also disclosed are flavor compositions and consumer products having the hydrolyzed pectin.

### BACKGROUND

Pectin is a complex colloidal heterogeneous polysaccharide abundant in the primary cell wall and middle lamella of fruits and vegetables. It is a dietary fiber contributing beneficial effects such as improved digestion and lowering of blood cholesterol. It is also a food additive primarily used for its gelling and stabilizing properties in food applications such as jams, jellies, confectionery, fruit beverages, and desserts. Emulsion stabilizing properties of pectin has been of increasing interest to researchers when comparing with other commonly found emulsifying biopolymers such as gum arabic and proteins. The potential use of pectin as an emulsifier and stabilizer to encapsulate lipophilic compounds is particularly important for flavors and fragrances as consumers nowadays desire natural and recognizable ingredients on the product label declaration.

Among different sources of pectin such as citrus peel, apple pomace, or sugar beet pulps, pectin extracted from sugar beet has been reported as a stronger emulsifier from its high contents of acetyl groups and protein. However, the viscosity of sugar beet pectin upon hydration has limited its use in emulsion due to its large molecular size. Several prior art discloses methods to produce pectin hydrolysis products (hydrolysates) to reduce the molecular size: U.S. Pat. No. 5,472,952, U.S. Pat. No. 8,435,958, U.S. Pat. No. 9,113,650, and U.S. Pat. No. 2016/00160248. Nevertheless, U.S. Pat. No. 5,472,952 concerns mainly about the nutritional compositions of pectin hydrolysates. U.S. Pat. No. 8,435,958 concerns about the reduction and/or prevention of the adhesion of pathogenic substances and organisms to eukaryotic cells. US 2016/0016248 concerns about the pulverulent ceramide from sugar beet pulp by an enzymatic reaction. U.S. Pat. No. 9,113,650 discloses methods of a pectic enzyme treated pectin and numerous applications, where this invention limits to the specific citrus pectin and a hydrolysate from a complete hydrolysis.

There is a need for a natural emulsifier that can be used to prepare clear liquid beverage concentrates and ready-to-drink beverages.

### SUMMARY

The present invention is based on the discovery of certain hydrolyzed pectin prepared from a natural process and useful as an emulsifier.

Accordingly, one aspect of this invention relates to a process of hydrolyzing a pectin. The method includes the steps of: (a) providing a pectin, and (b)hydrolyzing the pectin in a reaction mixture containing a pectinase to obtain a hydrolyzed pectin.

In some embodiments, the hydrolyzing step is performed (i) at a temperature of 10 to 70° C., preferably 15 to 55° C., more preferably 40 to 50° C.; (ii) at a pH of 1 to 10, preferably, 2 to 7, and more preferably 4 to 5; and/or (iii) for 0.5 to 48 hours, preferably 0.5 to 10 hours, more preferably 1 to 5 hours. The pH of the reaction mixture can be adjusted with an inorganic or organic acid such as citrus acid to a pH of 1 to 7, preferably 2 to 6, and more preferably 3 to 5.

The process of this invention optionally includes the step of separating the hydrolyzed pectin from the reaction mixture to obtain a purified hydrolyzed pectin. Separation is achieved through conventional separation technology such as ultrafiltration and lyophilization.

Any types of pectin is suitable to be hydrolyzed using the process. An example is a sugar beet pectin.

The pectin is reacted to obtain a hydrolyzed pectin having a molecular weight of 1000 to 80000 Daltons with an upper limit of 75000, 70000, 60000, 50000, 45000, 40000, 30000, 25000, 20000, 10000, 8000, 7500, or 6000 and a lower limit of 1500, 2000, 2500, 3000, 4000, or 5000. Such hydrolyzed pectin can have an averaged degree of esterification (DE) of 15 to 80%.

Suitable pectinases include Viscozyme L (Aspergillus sp.) and Pectinex Ultra SP-L (Aspergilus aculeatus), which can be present at a level of at least 0.0002% by weight of the reaction mixture, preferably, 0.005 to 5%.

Another aspect of this invention relates to a hydrolyzed pectin prepared from any one of the processes described above.

Also within the scope of this invention is an emulsifier system comprising the hydrolyzed pectin described above.

This emulsifier system is useful to prepare flavor compositions. Accordingly, this invention also contemplates a flavor composition contains the emulsifier system. The flavor composition can be in a powder form prepared by a spray drying process and optionally contains one or more carbohydrates. In some embodiments, the flavor composition is in an emulsion form and optionally contains a weighting agent, wherein the flavor composition. In other embodiments, the flavor composition contains a microcapsule having a microcapsule core and a microcapsule wall encapsulating the microcapsule core, the microcapsule core contains the flavor, and the microcapsule wall is formed of an encapsulating polymer. Optionally, the encapsulating polymer is gelatin, gum arabic, polysiloxane, or a combination thereof. In some embodiments, the flavor composition is in a granule form further containing one or more carbohydrates, one or more anticaking agent, one or more absorbents, or a combination thereof.

The emulsifier system is also useful in preparing microcapsule compositions. A typical microcapsule composition contains a microcapsule prepared in the presence the emulsifier system. The microcapsule contains a microcapsule core and a microcapsule wall encapsulating the microcapsule core. The microcapsule core contains a fragrance, a flavor, a malodor counteracting agent, or any other active materials such as pro-fragrance, vitamin or derivative thereof, anti-inflammatory agent, fungicide, anesthetic, analgesic, antimicrobial active, anti-viral agent, anti-infectious agent, anti-acne agent, skin lightening agent, insect repellant, animal repellent, vermin repellent, emollient, skin moisturizing agent, wrinkle control agent, UV protection agent, fabric softener active, hard surface cleaning active, skin or hair conditioning agent, flame retardant, antistatic agent, nanometer to micron size inorganic solid, polymeric or elastomeric particle, taste modulator, cell, probiotic, and any combination thereof.

The microcapsule wall is formed of an encapsulating polymer, which can be selected from the group consisting of a polyacrylate, polyurea, polyurethane, polyacrylamide, polyester, polyether, polyamide, poly(acrylate-co-acrylamide), starch, silica, gelatin and gum Arabic, alginate, chitosan, polylactide, poly(melamine-formaldehyde), poly(urea-formaldehyde), and combination thereof.

Still within the scope of this invention is a consumer product that contains any of the flavor or fragrance compositions described above.

Exemplary consumer products include a shampoo, a hair conditioner, a hair rinse, a hair refresher, a hair fixative or styling aid, a hair bleach, a hair dye or colorant, a soap, a body wash, a cosmetic preparation, an all-purpose cleaner, a bathroom cleaner, a floor cleaner, a window cleaner, a bath tissue, a paper towel, a disposable wipe, a diaper rash cream or balm, a baby powder, a diaper, a bib, a baby wipe, an oral care product, a tooth paste, an oral rinse, an tooth whitener, a denture adhesive, a chewing gum, a breath freshener, an orally dissolvable strips, a chewable candy, a hard candy, a hand sanitizer, an anti-inflammatory balm, an anti-inflammatory ointment, an anti-inflammatory spray, a health care device, a dental floss, a toothbrush, a tampon, a feminine napkin, a personal care product, a sunscreen lotion, a sunscreen spray, a wax-based deodorant, a glycol type deodorant, a soap type deodorant, a facial lotion, a body lotion, a hand lotion, a body powder, a shave cream, a bath soak, an exfoliating scrub, a foot cream, a facial tissue, a cleansing wipe, a fabric care product, a fabric softener, a fabric refresher, an ironing water, a liquid laundry detergent, a liquid dish detergent, an automatic dish detergent, a unit dose tablet or capsule, a scent booster, a drier sheet, a fine fragrance, a solid perfume, a powder foundation, a liquid foundation, an eye shadow, a lipstick or lip balm, an Eau De Toilette product, a deodorant, a rug deodorizer, a candle, a room deodorizer, a disinfectant, an anti-perspirant, an roll-on product, and an aerosol product. Additional Examples of the consumer products are consumer product is selected from the group consisting of baked goods, dairy products, fruit ices, confectionery products, sugarless candies, jams, jellies, gelatins, puddings, animal feeds, pressed confectionery tablets, hard-boiled candies, pectin-based candies, chewy candies, creme-centered candies, fondants, sugarless hard-boiled candies, sugarless pectin-based candies, sugarless chewy candies, sugarless creme-centered candies, toothpastes, mouthwashes, breath fresheners, carbonated beverages, mineral waters, powdered beverage mixes, non-alcoholic beverages, cough drops, lozenges, cough mixtures, decongestants, anti-irritants, antacids, anti-indigestion preparations and oral analgesics, and chewing gums.

The details of one or more embodiments of the invention are set forth in the description below. Other features, objects, and advantages of the invention will be apparent from the description and the claims.

### DETAILED DESCRIPTION

The hydrolyzed pectin is an ideal emulsifier useful in preparing flavor emulsions, clear liquid beverages, liquid beverage concentrates, spray dried flavor compositions, flavor granules, microcapsules containing flavors or fragrances. This emulsifier demonstrates high emulsification capacity and emulsification stability.

In particular, a hydrolyzed sugar beet pectin has emulsification and stabilization properties in both flavor and fragrance microcapsule compositions in either a dry form or a slurry form. These microcapsule compositions can be used in various consumer and food products including fabric conditioner, detergent, shampoo, body wash, beverages, dairy products, baked products, and confectionary products such as candies and chewing gum.

The hydrolyzed pectin has a potential to improve the shelf life of flavor and/or fragrance compositions.

Additionally, the hydrolyzed pectin obtained by an enzymatic reaction is deemed natural and therefore more acceptable to consumers than synthetic emulsifiers.

Suitable flavors may be chosen from synthetic flavors, flavoring oils and oil extracts derived from plants, leaves, flowers, fruits, and combinations thereof. Representative flavor oils include, but are not limited to, spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit, and fruit essences including apple, pear, peach, strawberry, watermelon, raspberry, cherry, plum, pineapple, apricot and so forth. These flavors can be used individually or in admixture.

These flavors may include volatile compounds such as acetaldehyde, dimethyl sulfide, ethyl acetate, ethyl propionate, methyl butyrate, and ethyl butyrate. Flavors containing volatile aldehydes or esters include, e.g., cinnamyl acetate, cinnamaldehyde, citral, diethylacetal, dihydrocarvyl acetate, eugenyl formate, and p-methylanisole. Further examples of volatile compounds that may be present in the flavor oils include acetaldehyde (apple); benzaldehyde (cherry, almond); cinnamic aldehyde (cinnamon); citral, i.e., alpha citral (lemon, lime); neral, i.e., beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e., trans-2 (berry fruits); tolyl aldehyde (cherry, almond); veratraldehyde (vanilla); 2,6-dimethyl-5-heptenal, i.e., melonal (melon); 2-6-dimethyloctanal (green fruit); and 2-dodecenal (citrus, mandarin); cherry; or grape and mixtures thereof. The composition may also contain taste modulators and artificial sweeteners.

The physical, chemical, and odor properties of exemplary volatile compounds are presented in Table 1.

TABLE 1

| | | | Water | |
|---|---|---|---|---|
| | | Boiling | Solubility | |
| | MW | Point | (g/L, | |
| Compound | (g/mol) | (° C.) | approx.) | Odor Descriptors* |
| acetaldehyde | 44.05 | 21 | Soluble | pungent; ethereal |
| dimethyl sulfide | 62.02 | 36 | Insoluble | cabbage |
| ethyl acetate | 88.11 | 77 | 90 | ethereal; fruity |
| ethyl propionate | 102.13 | 99 | 14 | sweet; fruity; ethereal |
| methyl butyrate | 102.13 | 102 | 15 | fruity; pineapple |
| ethyl butyrate | 116.16 | 121 | 6 | fruity; pineapple |

*The Good Scents Company and Merck Index, 12^{th} Ed.

In general, the flavor composition contains a flavor oil concentration of 0.01 to 90% with an upper limit of 80%, 70%, 60%, 50%, 40% and 30% and a lower limit of 0.05%, 0.1%, 0.5%, 1%, 2%, 5%, and 10%. In certain embodiments, the flavor composition contains a flavor oil between 0.1 to 20%. In other embodiments, the flavor composition contains a flavor oil between 1 to 5%.

The flavor composition can contain a water phase and one or more adjuvants such as a viscosity control agent, a density modifier, a stabilizer, a solubilizing agent, and a pH modifier.

Exemplary viscosity control agents are natural or modified carbohydrate gums including seaweed extracts such as agar, algins, and carrageenans; plant seed gums such as locust bean gum and guar gum; polysaccharide gums such as xanthan gum; plant exudates such as gum arabic, gum tragacanth, and gum karaya; and, synthetic gums such as sodium carboxymethylcellulose, hydroxypropylmethylcellulose, methylcellulose, and microcrystalline cellulose. Other suitable viscosity control agents, which may be polymeric or colloidal, include modified cellulose polymers such as methylcellulose, hydoxyethylcellulose, hydrophobically modified hydroxyethylcellulose, and cross-linked acrylate polymers such as Carbomer, hydrophobically modified polyethers. Optionally, silicas, either hydrophobic or hydrophilic, can be included at a concentration from 0.01 to 20%, more preferable from 0.5 to 5%, by the weight of the emulsion composition. Examples of hydrophobic silicas include silanols, surfaces of which are treated with halogen silanes, alkoxysilanes, silazanes, and siloxanes, such as SIPERNAT D17, AEROSIL R972 and R974 available from Degussa. Exemplary hydrophilic silicas are AEROSIL 200, SIPERNAT 22S, SIPERNAT 50S (available from Degussa), and SYLOID 244 (available from Grace Davison). Nonlimiting examples of a solubilizing agent include surfactants (e.g., SLS and

Tween 80), acidic compounds (e.g., mineral acids such as sulfuric acid, hydrochloric acid, nitric acid, and phosphoric acid, and carboxylic acids such as acetic acid, citric acid, gluconic acid, glucoheptonic acid, and lactic acid), basic compounds (e g , ammonia, alkali metal and alkaline earth metal hydroxides, primary, secondary, or tertiary amines, and primary, secondary, or tertiary alkanolamines), ethyl alcohol, glycerol, glucose, galactose, inositol, mannitol, glactitol, adonitol, arabitol, and amino acids.

Suitable density modifiers include hydrophobic materials and materials having a desired molecular weight (e.g., higher than 12,000), such as silicone oils, petrolatums, vegetable oils, especially sunflower oil and rapeseed oil, and hydrophobic solvents having a desired density (e.g., less than 1,000 Kg/m³ at 25° C., such as limonene and octane. See WO 2000/059616, EP 1 502 646, and EP 2 204 155.

In some embodiments, a stabilizer (e.g., a colloidal stabilizer) is added to stabilize the flavor composition. Examples of colloidal stabilizers are polyvinyl alcohol, cellulose derivatives such hydroxyethyl cellulose, polyethylene oxide, copolymers of polyethylene oxide and polyethylene or polypropylene oxide, or copolymers of acrylamide and acrylic acid.

In some embodiments, one or more pH modifiers are included in the water phase to adjust the pH value of the flavor composition. The pH modifiers can also assist in stabilizing the emulsion or achieving a desired taste. Exemplary pH modifiers are metal hydroxides (e.g., LiOH, NaOH, KOH, and Mg(OH)₂), metal carbonates and bicarbonates (e.g., CsCO₃, Li₂CO₃, K₂CO₃, NaHCO₃, and CaCO₃), metal phosphates/hydrogen phosphates/dihydrogen phosphates, metal sulfates, ammonia, mineral acids (HCl, H₂SO₄, H₃PO₄, and HNO₃), carboxylic acids (e.g., acetic acid, citric acid, lactic acid, benzoic acid, and sulfonic acids), and amino acids. A skilled person in the art can determine which pH modifier to use and how much to use so that the pH of the flavor composition is adjusted to 1-9 (e.g., 1-7, 1-5, 2-5, 1-4, and 1-3).

The emulsifier hydrolyzed pectin in a flavor or fragrance composition can be present at a level from 0.01 to 20% by weight of the composition with an upper limit of 18%, 15%, 12%, 10%, 8%, 6%, or 5% and a lower limit of 0.02%, 0.05%, 0.1%, 0.2%, 0.5%, or 1% (e.g., 0.05 to 10%, 0.2 to 5%, and 1-3%). The amount is determined by the amount of flavor oil, the flavor oil droplet size, and the existence of an adjuvant.

Typically, the weight ratio between the hydrolyzed pectin and the flavor oil is 1:100 to 100:1, preferably, 1:10 to 10:1, and more preferably, 1:5 to 5:1.

In some aspects of this invention, the emulsifier system containing the hydrolyzed pectin also contains one or more co-emulsifiers (e.g., one co-emulsifier, two co-emulsifiers, and three co-emulsifiers). In some embodiments, the hydrolyzed pectin is used without a co-emulsifier. Namely, the flavor composition is free of a co-emulsifier.

Exemplary co-emulsifiers are standard lecithins, fractioned lecithins, polyoxyethylene sorbitan fatty acid esters, ammonium phosphatides, mono- or diglycerides of fatty acids including distilled monoglycerides, acetic acid esters of mono- and diglycerides (Acetem), lactic acid esters of mono- and diglycerides of fatty acids (Lactem), citric acid esters of mono and diglycerides of fatty acids (Citrem), mono and diacetyl tartaric acid esters of mono and diglycerides of fatty acids (Datem), succinic acid esters of monoglycerides of fatty acids (SMG), ethoxylated monoglycerides, sucrose esters of fatty acids, sucroglycerides, polyglycerol esters of fatty acids, polyglycerol polyricinoleate, propane-1,2 diol esters of fatty acids, thermally oxidized soya bean oil interacted with mono- or diglycerides of fatty acids, sodium stearoyl lactylate (SSL), calcium stearoyl lactylate (CSL), stearyl tartrate, sorbitan esters of fatty acids, polyglycerol esters of interesterified castor oil acid (E476), sodium stearoyllatylate, sodium lauryl sulfate and polyoxyethylene stearate such as for instance polyoxyethylene(8)stearate and polyoxyethylene(40)stearate, polyoxyethylated hydrogenated castor oil (for instance, such sold under the trade name CREMO-PHOR), block copolymers of ethylene oxide and propylene oxide (for instance as sold under the trade name PLURONIC or the trade name POLOXAMER), polyoxyethylene fatty alcohol ethers, and polyoxyethylene stearic acid ester. Examples of polyoxyethylene sorbitan fatty acid esters include polyoxyethylen sorbitan monolaurate (polysorbate 20), polyoxyethylen sorbitan monooleate (polysorbate 80), polyoxyethylen sorbitan monopalmitate (polysorbate 40), polyoxyethylen sorbitan monostearate (polysorbate 60), polyoxyethylen sorbitan tristearate (polysorbate 65). Examples of sorbitan esters of fatty acids are sorbitan monostearate, sorbitan tristearate, sorbitan monolaurate, sorbitan monooleate, sorbitan monopalmitate, and saccharose esters of fatty acids.

The flavor composition described above can be used in a variety of consumer, food, or pharmaceutical products. In particular, they find application in gums, confections, oral care products, beverages, snacks, dairy products, soups, sauces, condiments, cereals, and baked goods.

In some embodiments, the flavor composition is dosed at a level between 1 ppm to 60% (e.g., 1 ppm to 20% and 5 ppm to 5%) by weight of the final food product so that the product contains a flavor oil 0.01 ppm to 10% (0.1 ppm to 5%, 0.5 ppm to 1%, and 1 ppm to 100 ppm).

The flavor composition of this invention can be used in the following products:
a. Confectioneries, preferably selected from the group consisting of chocolate, chocolate bar products, other products in bar form, fruit gums, hard and soft caramels and chewing gum
   i. Gum
      1. Gum base (natural latex chicle gum, most current chewing gum bases also presently include elastomers, such as polyvinylacetate (PVA), polyethylene, (low or medium molecular weight) polyisobutene (PIB), polybutadiene, isobutene-isoprene copolymers (butyl rubber), polyvinylethylether (PVE), polyvinylbutyether, copolymers of vinyl esters and vinyl ethers, styrene-butadiene copolymers (styrene-butadiene rubber, SBR), or vinyl elastomers, for example based on vinylacetate/vinyllaurate, vinylacetate/vinylstearate or ethylene/vinylacetate, as well as mixtures of the mentioned elastomers, as described for example in EP 0 242 325, U.S. Pat. No. 4,518,615, U.S. Pat. No. 5,093,136, U.S. Pat. No. 5,266,336, U.S. Pat. No. 5,601,858 or U.S. Pat. No. 6,986,709.) 20-25%
      2. Powdered sugar 45-50%
      3. glucose 15-17%
      4. starch syrup 10-13%
      5. plasticizer 0.1%
      6. flavor 0.8-1.2%
      The components described above were kneaded by a kneader according to the foregoing formulation to provide a chewing gum. Encapsulated Flavor or sensate is then added and blended till homogeneous.
   ii. Breath Fresheners
   iii. Orally Dissolvable Strips
   iv. Chewable Candy
   v. Hard Candy
b. Baked products, preferably selected from the group consisting of bread, dry biscuits, cakes and other cookies;
c. snack foods, preferably selected from the group consisting of baked or fried potato chips or potato dough products, bread dough products and corn or peanut-based extrudates;
   i. Potato, tortilla, vegetable or multigrain chips
   ii. Popcorn
   iii. Pretzels
   iv. Extruded stacks
d. Cereal Products preferably selected from the group consisting of breakfast cereals, muesli bars and precooked finished rice products
e. Spice blends and consumer prepared foods
   i. Powder gravy, sauce mixes
   ii. Condiments
   iii. Fermented Products
f. Ready to heat foods: ready meals and soups, preferably selected from the group consisting of powdered soups, instant soups, precooked soups
   i. Soups
   ii. Sauces
   iii. Stews
   iv. Frozen entrees
g. Dairy Products milk products, preferably selected from the group consisting of milk beverages, ice milk, yogurt, kefir, cream cheese, soft cheese, hard cheese, powdered milk, whey, butter, buttermilk and partially or fully hydrolyzed milk protein-containing products Flavored milk beverages
   i. Yoghurt
   ii. Ice cream
   iii. Bean Curd
   iv. Cheese
h. Soya protein or other soybean fractions, preferably selected from the group consisting of soya milk and products produced therefrom, soya lecithin-containing preparations, fermented products such as tofu or tempeh or products produced therefrom and soy sauces;
i. meat products, preferably selected from the group consisting of ham, fresh or raw sausage preparations, and seasoned or marinated fresh or salt meat products
j. Eggs or egg products, preferably selected from the group consisting of dried egg, egg white and egg yolk
k. and oil-based products or emulsions thereof, preferably selected from the group consisting of mayonnaise, remoulade, dressings and seasoning preparations
l. fruit preparations, preferably selected from the group consisting of jams, sorbets, fruit sauces and fruit fillings; vegetable preparations, preferably selected from the group consisting of ketchup, sauces, dried vegetables, deep-frozen vegetables, precooked vegetables, vegetables in vinegar and preserved vegetables.

All parts, percentages and proportions refer to herein and in the claims are by weight unless otherwise indicated.

The values and dimensions disclosed herein are not to be understood as being strictly limited to the exact numerical values recited. Instead, unless otherwise specified, each such value is intended to mean both the recited value and a functionally equivalent range surrounding that value. For example, a value disclosed as “50%” is intended to mean “about 50%.”

All publications recited herein are incorporate by reference in their entirety.

The following examples are provided as specific embodiments of the present invention.

### EXAMPLE 1

### Preparation of Hydrolyzed Pectin

A hydrolyzed pectin, i.e., HP-1, can be obtained by hydrolyzing through an enzymatic reaction of Genu Beta pectin (commercially available from CP Kelco, Atlanta,

Georgia) in a reaction mixture containing 5 to 95 wt % (e.g., 10 wt %, 20 wt %, 30 wt %, 50 wt %, 70 wt %, and 90 wt %) Genu Beta pectin and 0.002 to 10 wt % (e.g., 0.005 wt %, 0.1 wt %, 0.5 wt %, 1 wt %, and 2 wt %) pectinase in water. GENU BETA pectin is a polysaccharide derived from naturally occurring structural components in sugar beets. It has been used in dressings as a natural stabilizer in place of synthetic propylene glycol alginates.

The enzymatic reaction is performed at a temperature of 15 to 70° C. (e.g., 25° C., 35° C., 40° C., 45° C., 50° C., 55° C., and 65° C.) over a reaction time of 0.5 to 48 hours (e.g., 1 hour, 2 hours, 3 hours, 5 hours, and 10 hours). Upon completion of the hydrolysis, the hydrolyzed pectin is separated by ultrafiltration and lyophilization.

The pH of reaction mixture is adjusted to 1-7 (e.g., 1.5, 2, 2.5, 3, 3.5, 4, 4.5, 5, 5.5, and 6) with an acid solution (e.g., 10% citric acid solution).

In a solvent such as ethyl acetate, ethanol, and water, the hydrolyzed pectin can be further fractionated into a hydrolyzed pectin having a molecular weight of greater than 10,000 Daltons (e.g., 15,000, 20,000, 30,000, 40,000, 50,000, 100,000, 250,000, 500,000, and 1,000,000 Daltons), 1,000 to 10,000 Daltons (e.g., 1500, 2000, 2500, 3000, 4000, 5000, 6000, 7000, 8000, and 9000 Daltons), and less than 1000 Daltons (e.g., 750 and 500 Daltons). In some embodiments, the molecular weight of the hydrolyzed pectin has an upper limit of 1,000,000, 750,000, 500,000, 250,000, 200,000, 150,000, 100,000, 75,000, 50,000, 25,000, 20,000, 15,000, 10,000, 7500, 5000, 4000, 3000, or 2000, and a lower limit of 250, 500, 750, 1000, 2000, 3000, 4000, 5000, 7500, 10,000, 15,000, 20,000, 25,000, 50,000, 75,000, or 100,000.

The hydrolyzed pectin having a predetermined molecular weight can be obtained by adjusting the concentration of the pectin or pectinase, the pH of the reaction mixture, the reaction temperature, the reaction time, or any combination thereof.

### EXAMPLE 2

### Flavor Emulsions

Flavor emulsions can be prepared following the following representative procedure. An aqueous phase is obtained by dissolving 2 grams of sodium benzoate, 2 grams of potassium sorbate, and 3 grams of citric acid in 625 grams of water. Hydrolyzed pectin HP-1 (10 grams) and gum arabic (240 grams; commercially available from Nexira, Somerville, N.J.) are then added, followed by the addition of 55 grams of orange oil (commercially available from International Flavors and Fragrances, New York, N.Y.) and 55 grams of ester gum (commercially available from Pinova Inc., Brunswick, Ga.) to obtain a flavor mixture. A coarse emulsion is prepared by subjecting the flavor mixture to a Silverson high-speed mixer at 6500 rpm until a mean particle size of 5 μm or below is achieved. The coarse emulsion is subsequently put through a Microfluidizer M110P (manufactured by Microfluidics, Westwood, Mass.) three times at 6500 psi to obtain a stable emulsion with the mean particle size of 0.8 um or below. The final emulsion is targeted to be stable at 25° C. for at least 6 months.

### EXAMPLE 3

### Flavor Granules

First, a free-flowing flavor powder is obtained by admixing 440 grams of corn dextrin (NUTRIOSE FM06, commercially available from Roquette America Inc., Geneva, Ill.), 20 grams of hydrolyzed pectin HP-1, and 100 grams of a mint flavor oil (International Flavors and Fragrances, New York, N.Y.). Subsequently, to the free-flowing flavor powder are added 240 grams of isomalt, 150 grams of modified food starch, and 50 grams of sorbitol to obtain a powder blend, which is then fed into a twin-screw extruder with the highest temperature zone set at 170° C. to form a hot melt. After extruding the hot melt through a die, the resultant strands are cooled on a belt and cut into granules.

### EXAMPLE 4

### Flavor Capsules

An aqueous phase is prepared by mixing 40 grams of tetraethoxysilane (TEOS) (commercially available from Evonik Corporation, Piscataway, N.J.) and 36 grams of 0.01 M hydrochloric acid aqueous solution at 45° C. for 30 minutes. Additional 376 grams of TEOS is added dropwise during a period of 1 hour. The resultant mixture is then stirred at 45° C. for 16 hours to obtain polysiloxane. Ethanol is formed as a byproduct which can be removed in a Rotavapor with the aid of 10 mmHg vacuum. The resultant 275 grams colorless liquid polysiloxane material (Poly-Si) is obtained and used in capsule preparation. The polysiloxane materials thus prepared have a viscosity of 5-30 (Brookfield DV1 viscometer, 60 rpm, ambient temperature, spindle 4).

After heating to 50° C. a solution of 2 grams gelatin A (commercially available from Great Lakes Gelatin Co., Grayslake, Ill.) in 118 grams of water, 20 gram s of 10% hydrolyzed pectin HP-1 aqueous solution is added, followed by the addition of 40 grams of flavor mint oil (International Flavors and Fragrances, New York, N.Y.) and 10 grams of Poly-Si. The pH is adjusted to 4.5 with a 10% acetic acid solution. The resultant mixture is stirred for 16 hours and then cooled slowly to 20° C. Subsequently, to the mixture was added a solution of 6 grams of sodium silicate (37.5% aqueous solution, commercially available from PQ Corp., Malvern, Pa.) in 80 grams of water. After the pH was adjusted to 6.6, the resultant mixture was stirred for 1 hour at room temperature to obtain a flavor capsule composition.

### EXAMPLE 5

### Spray Dried Flavor Composition

An emulsifier solution is obtained by dissolving 15 grams of hydrolyzed pectin HP-1 and 410 grams of maltodextrin M150 in 397 grams of water. Sucrose (50 grams) is added to the emulsifier solution, followed by the addition of 128 grams of strawberry flavor (commercially available from International Flavors and Fragrances, N.Y., N.Y.). A coarse emulsion is prepared by subjecting the solution to Silverson high-speed mixing at 6500 rpm until the mean particle size of 3 um or below is achieved. The coarse emulsion is then pumped into a conventional spray dryer with an inlet air temperature at 170-210° C. and an outlet temperature at 80-100° C. to result in free-flowing powders. The final spray dried flavor is stable at 25° C. for at least 12 months.

### EXAMPLE 6

### Fragrance Capsules

Ninety-six grams of a fragrance, Apple (commercially available from International Flavors and Fragrances, New York, N.Y.) is mixed with 24 grams of NEOBEE oil (Stepan, Chicago, Ill.) and 9.6 grams of isocyanate monomer, LUPRANATE 20 (BASF corporation, Wyandotte, Mich.) to form an oil phase. In a separate container, a 1% surfactant solution (160 grams) was prepared by dissolving 1.6 grams of hydrolyzed pectin HP-1 in water. The oil phase is then emulsified into the aqueous phase to form a fragrance emulsion under shearing (ULTRA TURRAX, T25 Basic, IKA WERKE) at 9500 rpm for 2 minutes.

The fragrance emulsion is placed in a round bottom vessel and 10.8 grams of 40% heamethylene diamine (HMDA) (INVISTA, Wichita, Kans.) is added under constant mixing with an overhead mixer. Formation of a fragrance capsule is visible by optical microscopy. The mixer speed is reduced after the addition of HMDS is complete. The capsule slurry is cured at 55° C. for 3 hours.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Indeed, to prepare a hydrolyzed pectin suitable as an emulsifier, one skilled in the art can select a suitable concentration for each of pectin and pectinase. Further, the reaction temperature, duration, and pH value can also be determined by a skilled artisan through assays known in the art.

From the above description, a skilled artisan can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A process of hydrolyzing a pectin, the process comprising the steps of:
providing a pectin, and
hydrolyzing the pectin in a reaction mixture containing a pectinase to obtain a hydrolyzed pectin.

2. The process of claim 1, wherein the hydrolyzing step is performed at a temperature of 10 to 70° C., preferably 15 to 55° C., more preferably 40 to 50° C. 10

3. The process of claim 1, wherein the hydrolyzing step is performed at a pH of 1 to 10, preferably, 2 to 7, and more preferably 4 to 5.

4. The process of claim 1, further comprising the step of separating the hydrolyzed pectin from the reaction mixture to obtain a purified hydrolyzed pectin.

5. The process of claim 4, wherein the separating is achieved through ultrafiltration and lyophilization.

6. The process of claim 1, wherein the pH of the reaction mixture is adjusted with citrus acid to a pH of 1 to 7, preferably 2 to 6, and more preferably 3 to 5.

7. The process of claim 1, wherein the pectin is a sugar beet pectin.

8. The process of claim 1, wherein the molecular weight of the hydrolyzed pectin is 1000 to 80,000 Daltons.

9. The process of claim 1, wherein the hydrolyzed pectin has a an averaged degree of esterification (DE) of 15 to 80% .

10. The process of claim 1, wherein the pectinase is Viscozyme L (Aspergillus sp.) and Pectinex Ultra SP-L (Aspergilus aculeatus) present at a level of at least 0.0002% by weight of the reaction mixture, preferably, 0.005 to 5%.

11. The process of claim 1, wherein the hydrolyzing step is performed for 0.5 to 48 hours, preferably 0.5 to 10 hours, more preferably 1 to 5 hours.

12. A hydrolyzed pectin prepared from the process of claim 1.

13. A emulsifier system comprising a hydrolyzed pectin of claim 12.

14. A flavor composition comprising the emulsifier system of claim 13 and a flavor.

15. The flavor composition of claim 14, further comprising one or more carbohydrates, wherein the flavor composition is in a powder form prepared by a spray drying process.

16. The flavor composition of claim 14, further comprising a weighting agent, wherein the flavor composition is in an emulsion form.

17. The flavor composition of claim 14, further comprising a microcapsule, wherein the microcapsule has a microcapsule core and a microcapsule wall encapsulating the microcapsule core, the microcapsule core contains the flavor, and the microcapsule wall is formed of an encapsulating polymer.

18. The flavor composition of claim 17, wherein the encapsulating polymer is gelatin, gum arabic, polysiloxane, or a combination thereof.

19. The flavor composition of claim 14, further comprising one or more carbohydrates, one or more anticaking agents, one or more absorbents, or a combination thereof, wherein the flavor composition is in a granule form.

20. A microcapsule composition comprising a microcapsule prepared in the presence of the emulsifier system of claim 13, wherein the microcapsule contains a microcapsule core and a microcapsule wall encapsulating the microcapsule core, the microcapsule core contains a fragrance, the microcapsule wall is formed of an encapsulating polymer.

21. The microcapsule composition of claim 20, wherein the encapsulating polymer is a polyacrylate, polyurea, polyurethane, polyacrylamide, polyester, polyether, polyamide, poly(acrylate-co-acrylamide), starch, silica, gelatin and gum Arabic, alginate, chitosan, polylactide, poly(melamine-formaldehyde), poly(urea-formaldehyde), or combination thereof.

22. A consumer product comprising a flavor composition of claim 14.

23. A consumer product comprising a flavor composition of claim 20.
